(19) United States
(12) Patent Application Publication
(10) Pub. No.: US 2022/0339460 A1
(43) Pub. Date: Oct. 27, 2022

(54) THERAPY SYSTEM

(71) Applicant: Archimedes Innovations, PBC, Washington, DC (US)

(72) Inventor: Peter Maine Forhan, Washington, DC (US)

(21) Appl. No.: 17/660,121

(22) Filed: Apr. 21, 2022

Related U.S. Application Data
(60) Provisional application No. 63/177,949, filed on Apr. 21, 2021.
(60) Provisional application No. 63/192,342, filed on May 24, 2021.

(51) Int. Cl. A61N 5/06

(52) U.S. Cl. CPC A61N 5/06; A61N 2005/0626; A61N 2005/0652; A61N 2005/0663; A61N 2005/066; A61N 2005/0632

(57) ABSTRACT

A therapy system includes a therapy output device that includes light sources and a controller. The light sources are configured to output electromagnetic radiation at different peak wavelengths. The controller is configured to operate the light sources to output the electromagnetic radiation to differ between a first therapy program and second therapy program by at least one of peak wavelengths, irradiance of one or more of the peak wavelengths, or energy density of the one or more peak wavelengths.

### CROSS-REFERENCE TO RELATED APPLICATION(S)

This application claims priority to and the benefit of U.S. Provisional Patent Application No. 63/177,949, filed Apr. 21, 2021, and U.S. Provisional Patent Application No. 63/192,342, filed May 24, 2021, the entire disclosures of which are incorporated by reference herein.

### TECHNICAL FIELD

This disclosure relates to therapy devices and, in particular, light-based therapy devices.

### BACKGROUND

Photobiomodulation, also referred to as low-level light therapy, applies electromagnetic radiation (or light) to surfaces of the body. More particularly, electromagnetic radiation of particular wavelengths may be applied to surfaces of the body to treat various biological conditions, such as musculoskeletal issues and hair loss, or otherwise induce biological responses.

Existing photobiomodulation therapy devices are typically used be healthcare professionals with particular expertise, while some consumer-focused devices are very limited in functionality and by user expertise.

### SUMMARY

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

In one implementation, an output device includes a plurality of light sources that are configured to output electromagnetic radiation at different peak wavelengths and a controller. The controller is configured to operate the light sources to output the electromagnetic radiation according to a first therapy program and a second program, each of the first therapy program and the second therapy program include parameters according to which the controller causes the light sources of the output device to output the electromagnetic radiation to differ between the first therapy program and the second therapy program by at least one of peak wavelength, power, irradiance, time, or energy density.

A method for providing photobiomodulation therapy with a therapy output device includes receiving, with the therapy output device, a first user input and a second user input. The first user input selects a first therapy program from a plurality of therapy programs. The first therapy program includes parameters according to which electromagnetic radiation is output by the therapy output device with a peak wavelength, an irradiance, and an energy density. Each of the other therapy programs of the plurality have one or more different parameters according to which electromagnetic radiation is output by the therapy output device with at least one of a different wavelength, a different irradiance, or a different energy density than the first therapy program. The second user input is to start the first therapy program. The method further includes operating the therapy output device to output the electromagnetic radiation according to the parameters.

The method may further include measuring, with a sensor of the therapy output device, a distance between the therapy output device and a user, and adjusting the irradiance according to the distance. The receiving of first user input may include receiving a signal from a user control device that is in wireless communication with the therapy output device.

According to an implementation, a therapy system includes a therapy output device that includes light sources and a controller. The light sources are configured to output electromagnetic radiation at different peak wavelengths. The controller is configured to operate the light sources to output the electromagnetic radiation to differ between a first therapy program and second therapy program by at least one of peak wavelengths, irradiance of one or more of the peak wavelengths, or energy density of the one or more peak wavelengths.

The therapy output device may be configured to communicate wirelessly with a user control device, and may configured to receive instructions from the user control device for the first therapy program and the second therapy program according to which the controller operates the light sources to output the electromagnetic radiation. The user control device may receive the instructions for the first therapy program and the second therapy program from a server. The therapy output device may receive updated instructions for the first therapy program from one of the server or the user control device, and the controller may operate the light sources according to the updated instructions to output the electromagnetic radiation to different from the first therapy program by at least one the different peak wavelengths, the irradiance of the one or more of the different peak wavelengths, or the energy density of the one or more the different peak wavelengths. The server may be configured to receive usage information from at least one of the therapy output device or the user control device, the usage information pertaining to usage of the therapy output device by a user. The usage information may include one of more of operation of the first therapy program or the second therapy program operated, duration of use, or distance between the therapy output device and the user. The therapy system may further include the server. The therapy system may further include the user control device.

The controller may be configured to operate the light sources according to the first therapy program to output the electromagnetic radiation at a first irradiance for a recommended duration, may be configured to receive a user-selected duration from a user that is less than the recommended duration, and may be configured to operate the light sources according the first therapy program and the user-selected duration to output the electromagnetic radiation at a second irradiance that is higher than the first irradiance for the user-selected duration.

The different peak wavelengths may include at least three peak wavelengths selected from a group consisting of a first peak wavelength of between 650 and 670 nanometers, a second peak wavelength of between 800 and 820 nanometers, a third peak wavelength of between 840 and 860 nanometers, and a fourth peak wavelength of between 1040 and 1060 nanometers. The different peak wavelengths may include the first peak wavelength, the second peak wavelength, the third peak wavelength, and the fourth peak wavelength. The light sources may be light-emitting diodes that each have only one of the peak wavelengths. The therapy output device may include a head that contains the light sources and includes a heat sink and an optical element through which the light sources output the electromagnetic radiation.

The therapy system may further include a grounding subsystem configured to conductively couple a user to ground. The grounding subsystem may include a user contact configured to electrically couple to a user, a grounding contact configured to electrically couple to the ground, and a conductive path extending therebetween the user contact and the grounding contact. The conductive path may extend through the therapy output device.

According to an implementation, a therapy output device includes a head and an arm. The head includes light sources that include first light sources that output electromagnetic radiation at a first peak wavelength in a red light spectrum and second light sources that output electromagnetic radiation at a second peak wavelength in an infrared light spectrum. The arm includes a first end coupled to the head and a second end, the arm being flexible to move the head relative to the second end and sufficiently rigid to support the head statically relative to the second end. The arm may be a gooseneck. The therapy output device may further include a base, the second end of the arm may be coupled to the base, and the base may be configured to rest on a flat surface and support the head and the arm thereabove.

According to an implementation, the therapy output device may include a controller and light sources. The light sources include one or more first light sources configured to output electromagnetic radiation at a first peak wavelength of between 650 and 670 nanometers, one or more second light sources configured to output electromagnetic radiation at second peak wavelength of between 800 and 820 nanometers, one or more third light sources configured to output electromagnetic radiation at a third peak wavelength of between 840 and 860 nanometers, and fourth light sources configured to output electromagnetic radiation at a fourth peak wavelength of between 1040 and 1060 nanometers. The controller operates the light sources according to first instructions of a first therapy program.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1A is a schematic view of a therapy system having a therapy output device and a user control device.

FIG. 1B is a schematic view of another configuration of light sources of a therapy output device of the therapy system of FIG. 1A.

FIG. 1C is a schematic view of a therapy system that includes a grounding subsystem.

FIG. 2 is a schematic view of an example hardware configuration of a controller of the therapy output device and/or the user control device.

FIG. 3A is a front view of an embodiment of the therapy output device.

FIG. 3B is a side view of the therapy output device of FIG. 3A.

FIG. 4A is a top, front, right perspective view of another embodiment of a therapy output device.

FIG. 4B is a top, rear, left perspective view of the therapy output device of FIG. 4A.

FIGS. 4C to 4H are front, rear, right, left, top, and bottom views of the therapy output device of FIG. 4A.

FIG. 5 is a schematic view of a network system having a plurality of therapy output devices and user control devices associated therewith.

FIG. 6 is a flowchart of a method of providing therapy with a therapy output device.

### DETAILED DESCRIPTION

Disclosed herein are various implementations of therapy systems and therapy output devices that are configured for photobiomodulation. More particularly, the therapy systems and therapy output devices disclosed herein are configured to output electromagnetic radiation at different wavelengths (or with different light sources), different strengths (or different irradiance), and different durations (or different energy densities) according to different predetermined and/or updated therapy programs and/or custom user-defined therapy programs. The predetermined therapy programs may, for example, be configured to provide different therapeutic outcomes or benefits according to scientific research. The predetermined therapy programs may further be updated according to new scientific research and/or feedback based on device usage and/or efficacy, which may be distributed via a network.

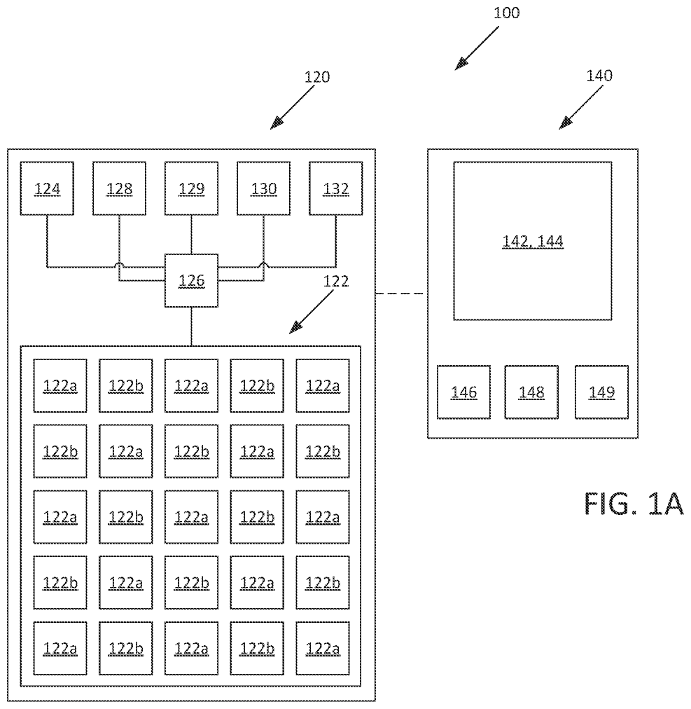

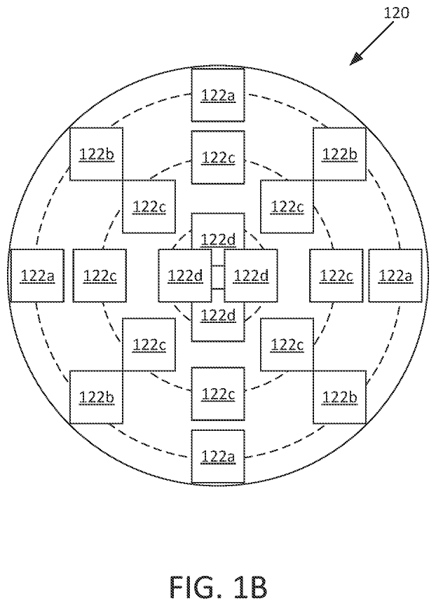

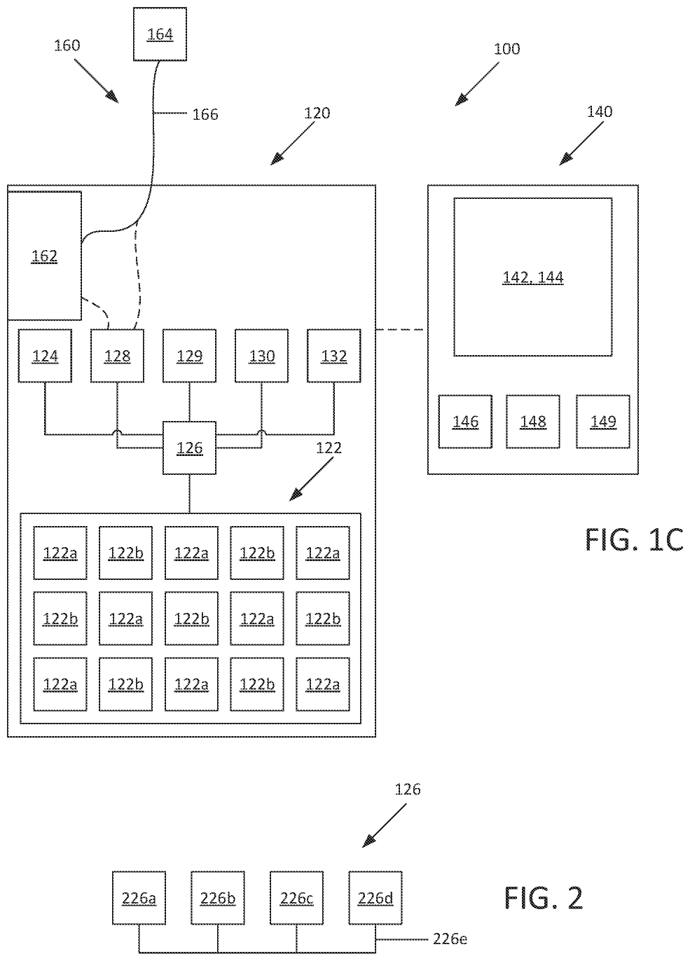

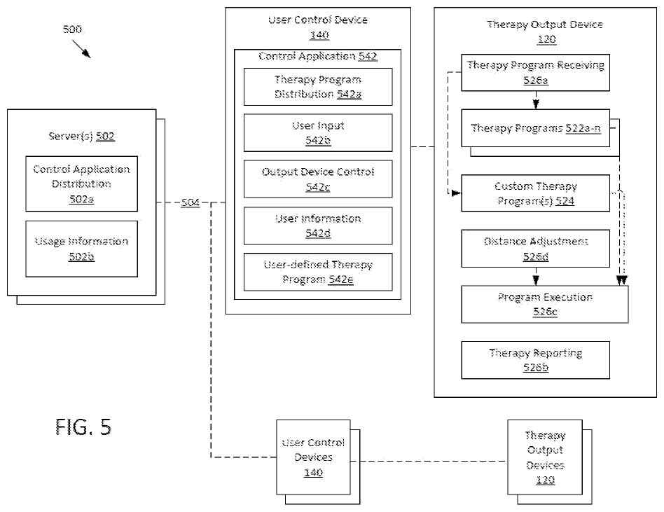

Referring to FIG. 1, a therapy system 100 generally includes a therapy output device 120 and a user control device 140. In some implementations, the therapy output device 120 may include the user control device 140 coupled thereto or otherwise incorporated therein. The therapy system 100 may also be referred to as a photobiomodulation system. The therapy output device 120 may also be referred to as photobiomodulation device or a polychromatic therapy device. The therapy system 100 may also be considered to include a network system 500 and/or a server 502 (as discussed below with reference to FIG. 5).

The therapy output device 120 is configured to output electromagnetic radiation and, in particular, electromagnetic radiation in one or more ranges of wavelengths, which are referred to herein as spectrums. For example, the therapy output device 120 may be configured to output the electromagnetic magnetic radiation at one or more peak wavelengths within one or more of a range of 400-500 nm (blue light spectrum), 600-700 nm (red light spectrum), or 700-1100 nm (infrared light spectrum). The therapy output device 120 may instead or additionally be configured to output the electromagnetic radiation at one or more additional peak wavelengths within one or more of a range of 500-600 nm (yellow light spectrum) or 290-320 nm (ultraviolet B or UVB spectrum). The different spectrums may also be identified numerically to distinguish therebetween (e.g., first and second spectrums).

The term “peak wavelength” refers to a wavelength at which the magnitude of the electromagnetic radiation output by the therapy output device 120 is at a singular or local maxima. The output magnitude of the electromagnetic radiation is, for example, irradiance that may be expressed as watts per square centimeter (W/cm\hat{ }2) at a defined distance from the therapy output device 120 (e.g., approximately 30 cm or other suitable distance that may be a recommended distance between the therapy output device 120 and the user). The therapy output device 120 may output electromagnetic radiation with any combination (e.g., one, two, three, four, or more) of peak wavelengths in the blue light spectrum of 400-450 nm (e.g., 410-420 nm, such as approximately 415 nm), in the red light spectrum of 630-680 nm (e.g., 650-670 nm, such as approximately 660 nm), and/or in the infrared light spectrum of 780-830 nm (e.g., 800-820 nm, such as approximately 805 nm), 825-875 nm (e.g., 840-860 nm, such as approximately 850 nm), and/or 1030-1080 nm (e.g., 1040-1060 nm, such as approximately 1050 nm). In the case of outputting electromagnetic radiation at multiple peak wavelengths, the different peak wavelengths may be identified according to the spectrum of the peak wavelength (e.g., blue light, red light, and/or infrared light peak wavelengths) and/or numerically between spectrums and/or within a spectrum (e.g., first and second peak wavelengths, or first and second infrared peak wavelengths). In one specific example, the therapy output device 120 is configured to selectively output the electromagnetic radiation at peak wavelengths in each of four spectrums that include a red light spectrum at 650-670 nm (e.g., 660 nm), a first infrared light spectrum at 800-820 nm (e.g., 810 nm), a second infrared light spectrum at 840-860 nm (e.g., approximately 850 nm), and a third infrared light spectrum at 1040-1060 nm (approximately 1050 nm). In another specific example, the therapy output device 120 is configured to selectively output the electromagnetic radiation at peak wavelengths in each of three spectrums that include a red light spectrum at 650-670 nm (e.g., 660 nm), a first infrared light spectrum at 840-860 nm (e.g., approximately 850 nm), and a second infrared light spectrum at 1040-1060 nm (approximately 1050 nm). In a still further example, the therapy output device 120 is configured to selectively output the electromagnetic radiation at peak wavelengths in each of seven spectrums that include one peak wavelength in each of the blue light spectrum, the yellow light spectrum, the red light spectrum, and the UVB spectrum and three peak wavelengths in the infrared spectrum (e.g., between 780-2500 nm, such as 780-1200 nm).

The therapy output device 120 includes one or more light sources 122, may include a sensor 124, and further includes a controller 126, and a power source 128. The one or more light sources 122 are individually or cooperatively configured to output the electromagnetic radiation in the different spectrums (e.g., at the one or more peak wavelengths). In one example, one or more of the one or more lights sources 122 are configured to each vary the peak output wavelengths of the electromagnetic radiation output thereby. That is, one or more of the light sources 122 are configured to output the electromagnetic radiation with different (i.e., more than one) peak wavelengths. In another example, one or more of the one or more light sources 122 are configured to output the electromagnetic radiation at one or more fixed peak output wavelengths, such as in the red light, the first infrared, the second infrared, or the third infrared light spectrum as described above. That is, each of the one or more light sources 122 is configured to output the electromagnetic radiation at one peak output wavelength, and different ones or groups of the light sources 122 may have different peak output wavelengths, such that operating different ones of the light sources 122 enables the therapy output device 120 to output the electromagnetic radiation at different peak output wavelengths.

The one or more light sources 122 may further be individually or cooperatively configured to output the electromagnetic radiation with different irradiance. In one example, the one or more light sources are configured to each vary irradiance of the electromagnetic radiation between different irradiance levels (e.g., having variable irradiance). For example, by increasing or decreasing the amount of power (e.g., current) provided to the one or more light sources 122, the irradiance may be increased or decreased, respectively. In another example, the one or more light sources 122 are configured to output the electromagnetic radiation at a fixed irradiance level, while the one or more light sources 122 may be selectively operated (e.g., turned on or off) for the therapy output device 120 to output the electromagnetic radiation at the different peak wavelengths with different (e.g., variable) irradiance.

The therapy output device 120 may, for example, be configured to output the electromagnetic radiation at one or more (e.g., four) of the peak wavelengths with irradiance at four inches (e.g., approximately ten centimeters) from the therapy output device 120 of at least the minimum irradiance and up to the maximum irradiance outlined in Table 1 below.

TABLE 1

| | Minimum Irradiance (mW/ | Maximum Irradiance (mW/ |
|---|---|---|
| Peak | cm\hat{ }2) Center Location/ | cm\hat{ }2) Center Location/ |
| Wavelength | Avg. 6″ radius (preferred | Avg. 6″ radius (preferred |
| (nanometers) | in parentheses) | in parentheses) |
| 650 to 670 | 1.0 (2.0)/0.5 (1.0) | 30 (20)/25 (20) |
| 800 to 820 | 1.0 (2.0)/0.5 (1.0) | 25 (18)/20 (15) |
| 840 to 860 | 1.0 (2.0)/0.5 (1.0) | 30 (20)/20 (15) |
| 1040 to 1060 | 0.5 (1.0)/0.1 (0.5) | 15 (10)/10 (5) |

For example, according to Table 1, the therapy output device 120 may include the light sources 122 that are capable of outputting electromagnetic radiation at a peak wavelength of between 650 and 670 nanometers measured at four inches from the therapy output device 120 of at least 1.0 mW/cm\hat{ }2 at a center illumination location (preferably at least 2.0 mW/cm\hat{ }2) and/or at least 0.5 mW/cm\hat{ }2 (preferably at least 1.0 mW/cm\hat{ }2) averaged over a 6 inch (e.g., 15 cm) radius and up to 30 mW/cm\hat{ }2 (preferably up to 20 mW/cm\hat{ }2) at a center illumination location and/or up to 25 mW/cm\hat{ }2 (preferably up to 20 mW/cm\hat{ }2) averaged over a 6 inch (e.g., 15 cm) radius.

Each of the light sources 122 may, for example, be a light emitting diode (LED) configured to output the electromagnetic radiation at or between different peak output wavelengths or at the one or more fixed peak output wavelengths. The light sources 122 may be arranged in a grid (e.g., with rows and columns of the light sources 122) or in a radiating pattern (e.g., with rings of increasing size and number of the light sources 122). For example, as shown in FIG. 1A, the light sources include first light sources 122a and second light sources 122b that are configured to output the electromagnetic radiation with a first peak wavelength and a second peak wavelength, respectively, that are different than each other (e.g., in the red light and infrared spectrums, respectively). While only two different light sources are illustrated in FIG. 1 (i.e., the first light sources 122a and the second light sources 122b), it should be understood that different numbers of the types of the light sources 122 may be provided (e.g., each of the light sources 122 having a different peak wavelength, such as one of three, four, five, or more different ones of the light sources 122 with different peak wavelengths). As referenced above and as illustrated in FIG. 1B, the light sources 122 may be arranged in a radiating pattern. For example, the light sources 122 (e.g., first, second, third, and fourth light sources 122a, 122b, 122c, 122d with different peak wavelengths) may be arranged in a ring-shaped patterns, for example, with three rings as shown (e.g., an outer ring, a middle ring, and an inner ring). Each of the rings may include one of the types of the light sources 122 (e.g., with one peak wavelength), as shown for the inner ring (including the fourth light sources 122d) and the middle ring (including the third light sources 122c), or may include more than of the types of the light sources 122 (e.g., including the first and second light sources 122a, 122b).

The therapy output device 120 is configured to selectively operate the one or more light sources 122 to output the electromagnetic radiation according to various different output parameters. The different output parameters may be predetermined and controlled according to a therapy program to achieve particular therapeutic goals or biological effect and may be further adjusted within such a therapy program according to various user criteria or other inputs. The therapy program may be referred to as a routine (e.g., a light routine). The therapy program may be part of a therapy regimen that includes operating the therapy program multiple times or a set of different therapy programs.

The different output parameters of a therapy program may include one or more peak wavelengths, power or irradiance (W/cm\hat{ }2), and time or energy density (J/cm\hat{ }2). The different output parameters may also be varied according to one or more inputs, which may be user selectable (e.g., duration), sensed (e.g., distance from the therapy output device 120 to the user), and/or biological (e.g., skin tone) that may be sensed or input by a user or other source. One or more of the parameters differ between different ones of the therapy programs. For example, a controller may be configured to operate the light sources to output the electromagnetic radiation to differ between a first therapy program and a second therapy program by at least one of the peak wavelengths, irradiance of one or more of the peak wavelengths (e.g., by controlling the amount of power supplied to the light sources 122), or energy density of the one or more peak wavelengths (e.g., by controlling the time during which the light sources 122 are operated). A therapy program may also be provided by a user in which the user provides the different output parameters described above.

The peak wavelength may be selectively controlled by varying the peak wavelength output by the light sources 122 having variable peak wavelengths output or by selectively operating different ones of the light sources 122 with fixed peak wavelengths (e.g., each of the light sources 122 may output electromagnetic radiation with only one peak wavelength).

The power refers to the amount power provided to the one or more light sources 122 at each of the wavelengths, which may be varied by selectively controlling electrical current supplied to the light sources 122 and/or by selectively controlling which of the light sources 122 the electrical current is supplied to. Based on the power supplied, the irradiance of the electromagnetic radiation output from the light sources 122 at the different wavelengths is varied, so as to achieve irradiance output for a given therapy program. Instead of power, the irradiance at the one or more peak wavelengths may be considered a parameter of the therapy program. The power may be generally constant or may vary over time, for example, in pulses. The power may also be varied according to a distance between the therapy output device 120 and the user, such as with the distance being detected with the sensor 124, so as to achieve a generally constant irradiance on the user.

The time refers to the amount of time that the electromagnetic radiation at the different peak output wavelengths at a given power or irradiance is output by the light sources 122. The time over which the electromagnetic radiation is output at a given irradiance (e.g., W/cm\hat{ }2) results in the energy density (e.g., J/cm\hat{ }2) at a given peak wavelength for a given therapy program. Thus, the time may be varied in order to achieve different energy density. Instead of time, the energy density at the one or more peak wavelengths may be considered a parameter of the therapy program. The energy density of a peak wavelength may also be referred to as a dose.

As referenced above, a therapy program may be varied according to one or more additional inputs, which may include duration, distance, and/or skin tone. For example, a therapy program may have a recommended irradiance and a recommended energy density to achieve a desired biological result, which may be based on scientific research. However, a user may not desire to dedicate the time (e.g., recommended time) required to achieve the recommended energy density with the recommended irradiance. Instead, the user may input a duration (e.g., a user-selected time) that is shorter than the recommended time, which may be within a predefined range, while the power is increased to output the electromagnetic radiation with higher irradiance to achieve the recommended energy density, or portion thereof, over the user-selected time. For example, the user may input a duration of between 1 and 30 minutes, such as between 2, 3, 4, 5 minutes or more and the recommended duration.

Further, a therapy program may have a recommended distance, measured between the therapy output device 120 and the user, for the recommended irradiance and the recommended energy density to achieve the desired biological result. However, if the therapy output device 120 is moved closer or further from that user than the recommended distance, the irradiance of the electromagnetic radiation will, respectively, become greater or lesser than the recommended irradiance, resulting, respectively, in greater or lesser than the recommended energy density. Instead, the therapy output device 120 may further include a distance sensor (e.g., a proximity sensor, such as the sensor 124, which may be an infrared sensor that operates at a wavelength greater than the highest peak wavelength of the light sources 122) that measures the distance between the therapy output device 120 and the user. The power is adjusted according to the distance to output the electromagnetic radiation, so as to achieve, or otherwise be nearer to, the recommended irradiance at the measured distance and also the recommended energy density than if the power were not adjusted according to the distance measurement. For example, as the distance measured by the sensor 124 between the sensor 124 and the user increases or decreases, the power supplied to the light sources 122 is increased or decreased, respectively, so as to increase or decrease the irradiance to account for the greater or lesser distance.

The controller 126 is configured to control the light sources 122 to output the light according to various different ones of the therapy programs, for example, controlling the power over the time supplied to the light sources 122 according to stored instructions and other inputs (e.g., user-selected time or distance). The controller 126 may be any suitable computing device or multiple computing devices. As shown in FIG. 2, the controller 126, in one example hardware configuration, may include a processor 226a, a memory 226b, a storage 226c, a communications interface 226d, and a bus 226e facilitating communication therebetween. The processor 226a may be any suitable processing device, such as a central processing unit, capable of executing instructions (e.g., software programming). The memory 226b is a short term, volatile storage device, such as a random access memory (RAM) unit. The storage 226c is a long term, non-volatile storage device, such as a hard disk drive (HDD) or solid state drive (SSD) capable of storing the instructions to be executed by the processor and which may include the one or more therapy programs. The storage 226c may be considered a computer- or machine-readable medium. The communications interface 226d is configured to send signals, such as control signals for supplying the power to the light sources 122, and receive signals thereto (e.g., from the sensor 124) and/or the user control device 140. While one example hardware configuration for the controller 126 is described, it should be understood that any other configuration of a controller may be utilized that is capable of perform the functions and methods and/or executing the instructions described herein.

The power source 128 is configured to supply power for operating the therapy output device 120 (e.g., the light sources 122, the sensor 124, and the controller 126). The power source 128 may, for example, include a plug that is configured to be inserted into and receive electrical power from a standard wall socket and/or may include a battery for storing electrical power. Instead or additionally, the power source 128 may be configured for inductive power transfer, for example, by including an inductive coil for receiving power to power the therapy output device 120 and/or to charge a battery. The power source 128 may, for example, be configured to supply sufficient power and at a suitable voltage for the light sources 122 to consume 20,000 mW, more, or less as may be required by the other components for the therapy program. The power source 128 may be further configured to condition electricity from another source for use by the therapy output device 120, the light sources 122, and/or other electrical components thereof, for example, converting by converting alternating current to direct current. Furthermore, the power source 128 may, as discussed in further detail below, be included or otherwise interact with a grounding subsystem 160.

The therapy output device 120 may further include one or more user inputs 130 and/or one or more displays 132. The user inputs 130, such as a physical button, are configured to receive physical inputs from the user (e.g., to turn on the therapy output device 120, select different ones of the therapy programs, and/or initiate different ones of the therapy programs). The one or more displays 132 are configured to provide a visual indication to the user, such as the therapy output device 120 being turned on, the selected therapy program, and/or remaining duration of the therapy program. The one or more displays 132 may, for example, include one or more lights (e.g., LEDs) that output visible light and/or a display screen. The one or more light sources 122 may also function as a visual indicator the therapy output device 120 is operating. For example, the light sources 122 may output the electromagnetic radiation at peak wavelengths in the visible light spectrum (e.g., blue, yellow, or red light spectrums) to provide a therapeutic function (e.g., with biologically significant irradiance and/or energy density), or may alternatively be operated only to provide a visual indication without a therapeutic function (e.g., without biologically significant irradiance and/or energy density) when only non-visible electromagnetic radiation is output for the therapeutic function (e.g., in the ultraviolet and/or infrared spectrums).

Referring additionally to FIG. 1C, the therapy system 100 may further include a grounding subsystem 160 that is configured to transfer electrons between the earth and the user (e.g., conductively). Without being bound to a particular theory, it is hypothesized that grounding the user may be beneficial to the benefits of the photobiomodulation or other therapeutic benefits provided by the therapy system 100 by providing electrons from ground that neutralize reactive oxygen species produced during photobiomodulation, as opposed to pulling electrons from and damaging other cells, which may limit the therapeutic benefit otherwise attainable. It is further hypothesized that grounding may replenish electrons released from mitochondria (e.g., from metals within the membrane of the mitochondria) due to the photoelectric effect of the electromagnetic radiation output by the therapy output device 120 to the user.

The grounding subsystem 160 generally includes a user contact 162 and a grounding contact 164 conductively coupled to the user contact 162 via a conductive path 166 extending therebetween and which may include various different conductors (e.g., conductive wire) and/or pass through other components of the therapy output device 120 (e.g., passing through or otherwise being associated with the power source 128, as indicated by the curved dashed line segments). The user contact 162 provides an electrically conductive surface for the user to contact (e.g., with the bare skin of their hand). The grounding contact 164 is configured to conductively couple to a ground (e.g., earth). For example, the grounding contact 164 may be a grounding pin of an alternating current (AC) power plug (e.g., a three-pin plug) and is received by a grounding receptacle of an AC power socket (e.g., a three-pin socket) that is in turn connected to ground (e.g., earth). In such case, the power plug may also be associated with the power source 128, for example, receiving electrical power from the AC power socket to operate the therapy output device 120. The conductive path 166 conductively couples the user contact 162 to the grounding contact 164 (e.g., for the transfer of electrons between the ground and the user). The user contact 162 and grounding contact 164 may also be referred to as electrodes.

The grounding subsystem 160 may be integrated with the therapy output device 120 (e.g., physically and/or electrically). Being physically integrated, an outer surface of a therapy output device 120 may include or otherwise be configured as the user contact 162 and/or the conductive path 166 may pass through the therapy output device 120. An outer surface or portion thereof of a handle 336, an arm 440, or a base 442 (each discussed in further detail below) may include or otherwise form the user contact 162, for example, by including a conductive polymer (e.g., a conductive rubber or faux leather), outer coating (e.g., paint), or other member (e.g., protrusion). Being electrically integrated, the grounding subsystem 160 and the therapy output device 120 may share common electrical components (e.g., the AC power plug, as referenced above).

Alternatively, the grounding subsystem 160 may be physically and/or electrically discrete from the therapy output device, for example, being provided as a separate peripheral (e.g., a grounding mat that is separately conductively coupleable to ground).

The therapy system 100 may also sense or otherwise collect data about the grounding subsystem 160, which may be referred to as grounding data. For example, the grounding subsystem 160 itself and/or the therapy output device 120 (when physically and/or electrically incorporated therein) may include a sensor (not shown) that detects whether the grounding subsystem 160 is connected to ground and/or a sensor that detects whether the user is touching and/or connected to the grounding subsystem 160. Instead or additionally, the user control device 140 may be configured to receive an input from the user indicating whether and/or when the user is and/or was using the grounding subsystem 160 (e.g., by presenting a button on a graphical user interface output thereby). The grounding data may, for example, be used to modify the therapy program (e.g., adjusting the output of different electromagnetic radiation, such as by wavelength and/or irradiance and/or the duration of the therapy program) and/or be collected to assess the various therapy programs (e.g., efficacy of therapy programs with and without grounding and/or usage of the grounding subsystem 160).

Referring again to FIG. 1A, the user control device 140 is configured to receive instructions from the user for controlling the therapy output device 120. The user control device 140 may, for example, be a handheld computing device (e.g., a smartphone or tablet computer). The user control device 140 generally includes a display screen 142, one or more user inputs 144, a controller 146, and a power source 148. The display screen 142 is configured to display a graphical user interface to the user. The one or more user inputs 144 may be virtual buttons displayed by the display screen 142 (e.g., incorporating the user inputs 144, such as being a capacitive touch screen) and/or physical buttons. The controller 146 is configured to receive signals thereto (e.g., from the user inputs 144, such as the touch screen) and/or from remote sources, such as from servers sending software programming with instructions for therapy programs to be sent to the therapy output device 120. The controller 146 may be configured as described for the controller 126, for example, with both of the controllers 126, 146 being configured to communicate with each other via any suitable wireless protocol, such as Bluetooth. For example, the therapy output device 120 may include a communications interface 129 (e.g., a Bluetooth radio), while the user control device 140 includes another communications interface 149 (e.g., a Bluetooth radio) by which the therapy output device 120 and the user control device 140 are in communication with each other. The therapy output device 120 and/or the user control device 140 may also be in communication with other devices, such as servers 502 and network devices (e.g., of a network 504) as discussed in further detail below (e.g., via the communications interfaces 129, 149, respectively).

The power source 148 may, for example, be a battery or wired power source. The power source 148 may be considered to further include appropriate electronics for conditioning power received and output thereby for use by the various electronics of the user control device 140 (e.g., a power converter for converting alternating current to direct current).

As discussed in further detail below, the user control device 140 may be configured to send therapy programs to the therapy output device 120, receive user inputs to select one or more therapy programs from among several therapy programs having different parameters, receive user inputs to start a therapy program, receive usage and feedback information, and transmit and/or receive corresponding signals to and/or from the therapy output device 120 and/or servers.

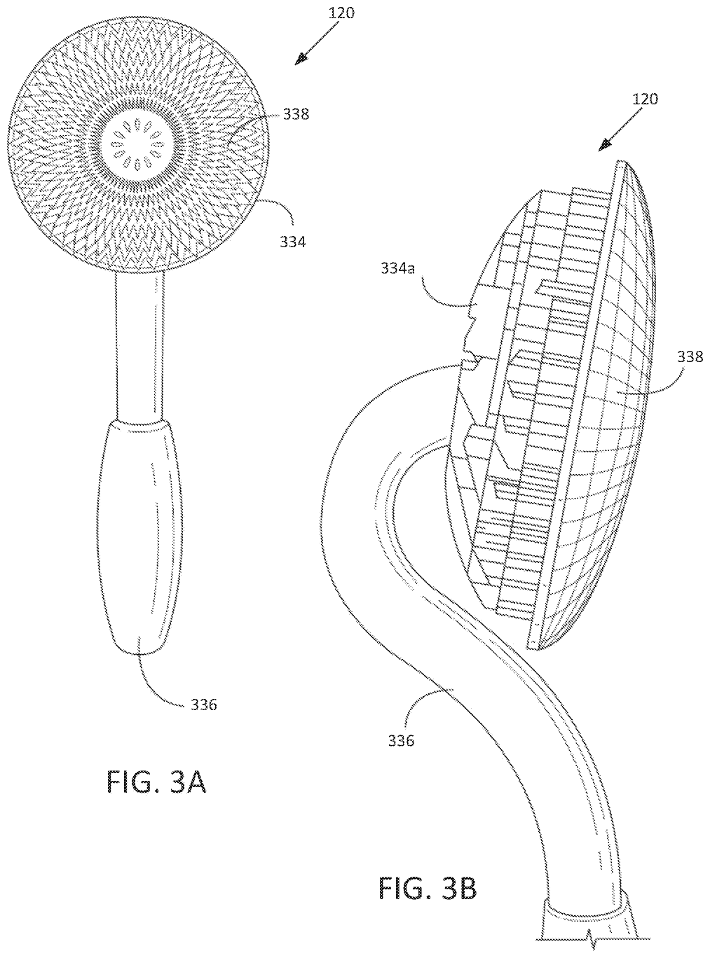

Referring to FIGS. 3A and 3B, in one example embodiment, the therapy output device 120 may generally include a body 334, a handle 336, and an optical element 338. The handle 336 is coupled to and extends from the body 334, allowing the user to hold the therapy output device 120 by the handle 336. The handle 336 may be removably coupleable to the body 334. The body 334 and the optical element 338 may be considered to cooperatively form a head of the therapy output device 120. The handle 336 may also be referred to as an arm.

The body 334 alone, or in conjunction with the handle 336, includes various electronic components described previously, including the light sources 122, the sensor 124, the controller 126, the power source 128, the user input 130, and/or the display 132. For example, the body 334 and/or the handle 336 are structures that define cavities that contain various of the electronic components and/or to which the various others of the electronic components may be coupled. For example, the body 334 may contain the light sources 122, the sensor 124, and the controller 126 coupled to a circuit board. The body 334 may also include a heat sink 334a that is configured to conduct heat away from the light sources 122 and other electronic components. The handle 336 may contain the power source 128. The controller 126 may instead be contained in the handle 336.

The optical element 338 is configured to obscure the electronics (e.g., the light sources 122) from view of the user. The optical element 338 may further be configured to diffuse the electromagnetic radiation emitted by the light sources 122. The optical element 338 may, for example, be configured for the electromagnetic radiation at the peak wavelengths to pass therethrough substantially unobstructed. The optical element 338 may be a singular component (e.g., formed monolithically or of multiple components coupled together) through which the light sources 122 emit the electromagnetic radiation at the different wavelengths (e.g., multiple ones of the lights sources 122 at each of the different wavelengths emit the electromagnetic radiation through the same optical element 338, such as only one optical element 338).

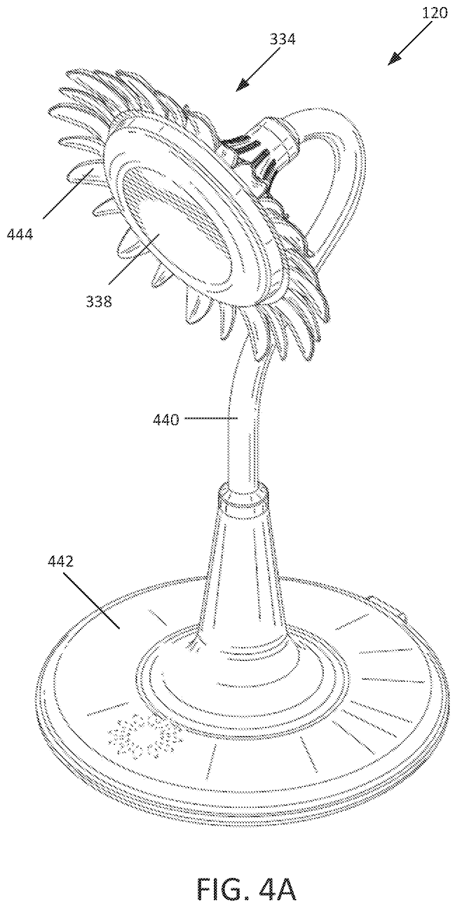

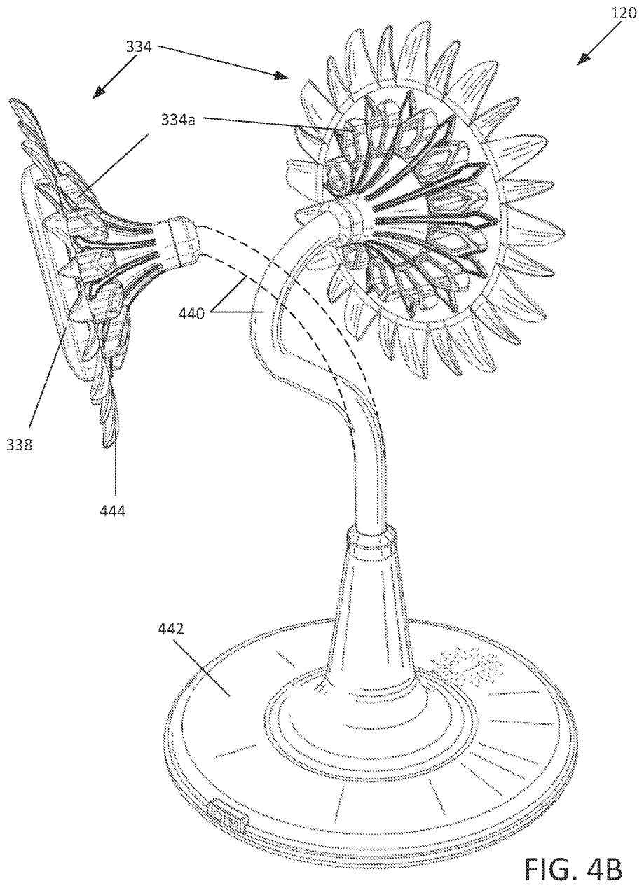

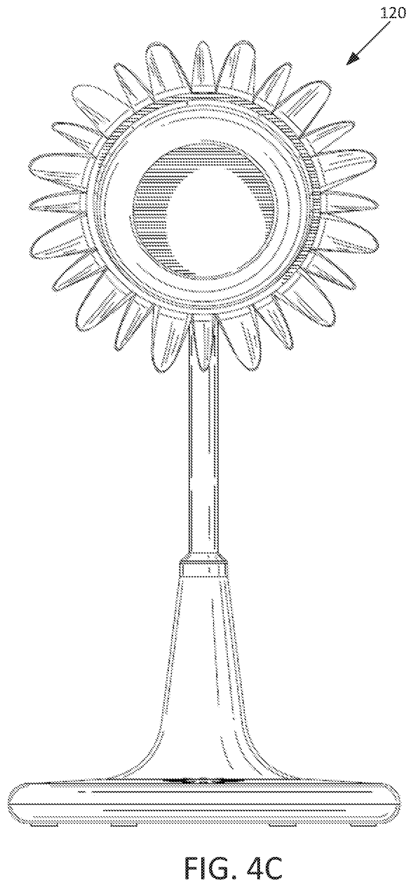

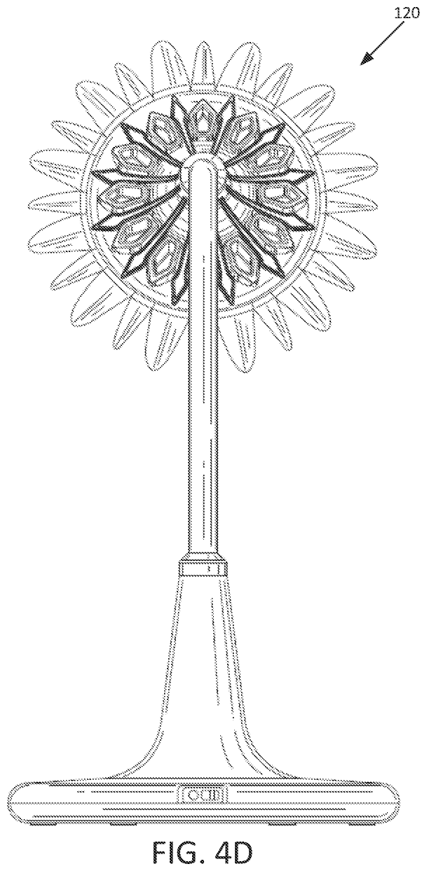

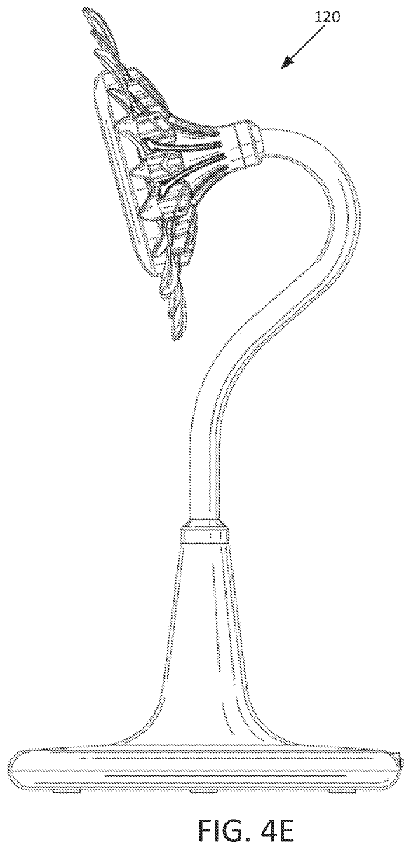

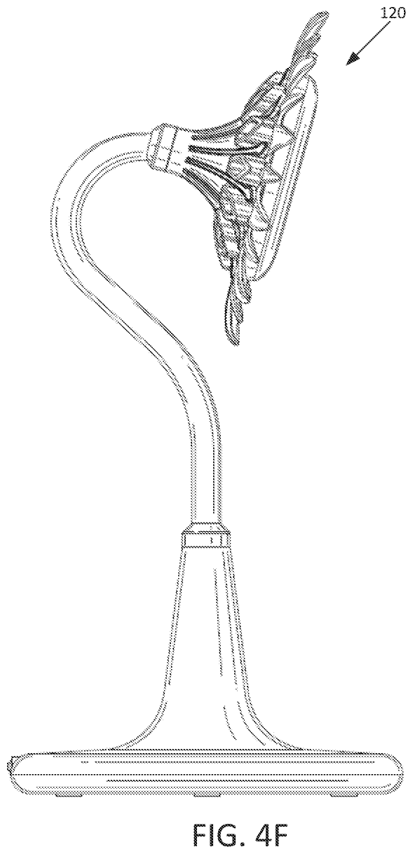

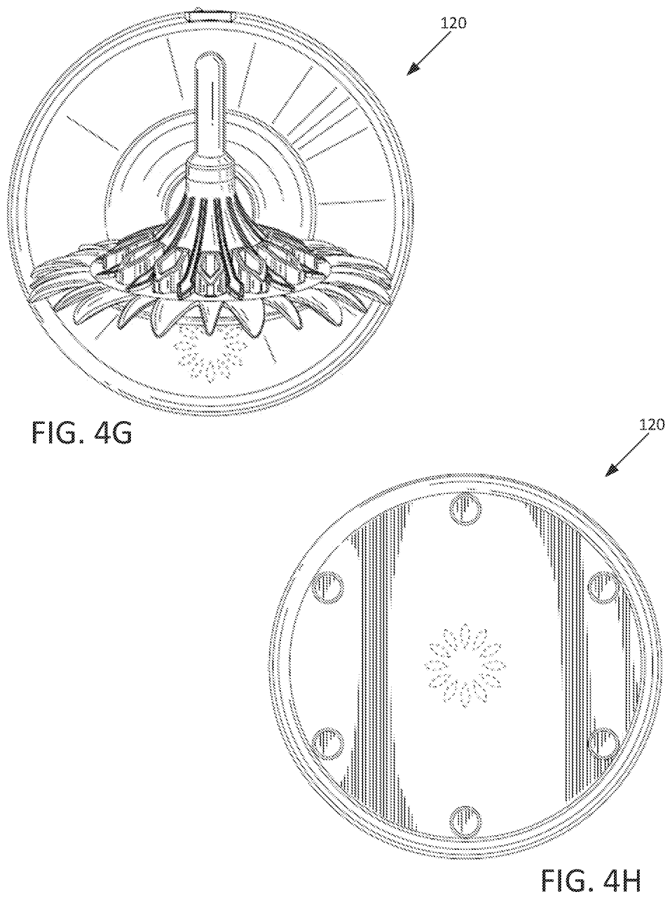

Referring to FIGS. 4A-4H, in another example embodiment, the therapy output device 120 may generally include the body 334, the optical element 338, an arm 440, and a base 442, and may further include a reflector 444. The body 334 and the optical element 338 may generally be configured as described previously (e.g., containing and/or including the light sources 122, sensor 124, heat sink 334a, and/or circuit board). The reflector 444 extends outward from the body 334 and may reflect electromagnetic radiation, first reflected from the user, back to the user. As shown, the reflector 444 is stylized as flower petals. The arm 440 extends from the base to the body 334 to support the body 334 there above. For example, the arm 440 may include a first end that is coupled to the body 334 (e.g., to the head) and a second end that may be coupled to the base 442.

The arm 440 may be movable (e.g., flexible) relative to the base 442 to move the body 334 into different positions and orientations relative to the second end thereof (e.g., relative to the base 442), as is illustrated in FIG. 4B with the body 334 being illustrated in a first position (i.e., the arm 440 being illustrated in solid lines) and a second position (i.e., the arm 440 being illustrated in dashed lines). For example, the arm 440 may include a tubular structure that is bendable therealong, while being sufficiently rigid to still support the body 334 in static positions and orientations relative to the base 442. The arm 440 may, for example, be configured as a gooseneck (e.g., a semi-rigid, coiled metal hose).

The base 442 is configured to support the arm 440 and the head (i.e., the body 334 and the optical element 338) thereabove. For example, the base 442 may have a flat bottom with a length and a width (e.g., being circular, as shown, ovoid, squared, rectangular, or other suitable shape) and weight (e.g., formed by a weighted material thereof) that stably supports the therapy output device 120 on a flat surface (e.g., a table or floor). The controller 126, the power source 128, and the communications interface 129 may be provided in a cavity of the base 442 and be electrically coupled to electronic components the body 334 (e.g., the light sources 122 and/or the sensor 124 thereof) conductive leads (e.g., wires) that extend through the arm 440 for operation thereof. RESUME

Referring to FIG. 5, a network system 500 is configured to provide and update therapy programs to the user control devices 140 and/or the therapy output devices 120 associated therewith and may be further configured to receive data from users via the user control devices 140. The therapy system 100 may be considered to include the network system 500. The network system 500 includes one or more servers 502 and a network 504 by which the one or more servers 502 communicate with the user control devices 140 and/or the therapy output devices 120 (directly or indirectly via the user control devices 140). For example, where each of the one or more servers 502 is or includes a hardware server, each of the servers 502 may be a computing device located at an operations center or a server device located in a rack, such as of a data center. The one or more servers 502 may, for example, be configured similar to the controller 126 (e.g., having a processor, memory, storage, communications interface, and bus), while also including other suitable hardware (e.g., a communications interface) by which the one or more servers 502 are in communication with the network 504 and, thereby, the user control devices 140 and/or the therapy output devices 120. The network 504 may, for example, be or include a local area network, a wide area network, a machine-to-machine network, a virtual private network, or another public or private network, or combinations thereof by which the one or more servers 502 are in communication with the user control devices 140 and/or the therapy output devices 120.

The one or more servers 502 may be configured to transmit software programs having executable instructions to the user control device 140 and/or to the therapy output devices 120, and may be further configured to receive usage and/or user feedback information therefrom. For example, the one or more servers 502 include, cooperatively or individually, a control application distribution module 502a and a usage information module 502b. The control application distribution module 502a includes instructions that are executable by the server 502 for sending a control application 542 to the user control devices 140. The control application 542 includes instructions executable by the user control devices 140 and, itself, may further include therapy program modules 522a-n that are transmitted to the therapy output device 120 and include instructions to be executed thereby to provide therapy to the user. For example, the therapy program modules 522a-n are or otherwise include instructions according to which the controller 124 of the therapy output device 120 operates the light sources 122 thereof to output the electromagnetic radiation. For example, the therapy output device 120 may communicate wirelessly with the user control device 140, such that the therapy output device 120 receives from and the user control device 140 sends thereto the instructions according to which the controller operates the light sources to output the electromagnetic radiation (e.g., the therapy program modules 522, or other instructions of first and second or more therapy programs). Alternatively, the therapy output device 120 may receive the therapy program modules 522a-n from the server 502 without the user control device 140.

It should be understood that the therapy program modules 522a-n include instructions that are executable by the therapy output device 120 to achieve the parameters of the therapy program (e.g., peak wavelengths, power and/or irradiance of the peak wavelengths, and time and/or energy density of the peak wavelengths), which may themselves be expressed in different manners suitable for controlling the light sources 122 of the therapy output device 120 (e.g., to control individual ones or groups of the light sources by supplying appropriate power thereto).

The control application distribution module 502a may also be configured to send updated versions of the control application 542 and/or updated therapy program modules 522a-n. The updated therapy program modules 522a-n may include instructions executable by the therapy output device 120 that include updated parameters (e.g., light sources or peak wavelengths, power or irradiance of the light sources or the peak wavelengths, and/or time or energy density of the light sources or the peak wavelengths) that vary from earlier versions of the therapy program modules 522a-n or updated instructions to achieve the updated parameters. Each of the updated therapy program modules 522a-n may replace an earlier version of the program module 522a-n. The updated therapy program modules 522a-n may be developed from new or updated research, or may be generated from the usage and/or user feedback information as discussed below.

The usage information module 502b includes instructions that are executable by the server 502 for receiving and/or processing usage information and/or user feedback information from the user control devices 140 and/or the therapy output devices 120. The usage information module 502b may be further configured to process the usage information and/or user feedback information and generate therewith updated therapy programs. Alternatively, usage information module 502b may collect and may transmit the usage information and/or user feedback information to another computing device, directly or indirectly, which in turn processes such information and generates therewith the updated therapy programs. In one example, machine learning may be used to update (e.g., optimize) the therapy program based on the usage information (e.g., duration and/or distance between the therapy output device 120 and the user, or other information that pertains to usage of the therapy output device 120 or a therapy program) or the user feedback information (e.g., user-reported information, which may include usability and/or efficacy information regarding the therapy program).

The user control devices 140 include the control application 542, which includes instructions that are executable by the controller 146 thereof. The control application 542 may be received by the user control device 140 from the one or more servers 502. The control application 542 may include, for example, a therapy program distribution module 542a, a user input module 542b, an output device control module 542c, and a usage information module 542d. The therapy program distribution module 542a is configured to receive one or more of therapy program modules 522a-n from the one or more servers 502 and send (e.g., transmit) one or more of the therapy program modules 522a-n to the therapy output device 120. For example, the user may select a particular therapy program with the user input module 542b, while the therapy program distribution module 542a transmits the therapy program module 522a of the selected therapy program to the therapy output device 120. The therapy program distribution module 542a may further be configured to change (e.g., adjust) the instructions of the therapy program modules 522a-n according to inputs received by the user input module 542b (e.g., adjusting instructions pertaining to power or irradiance and/or time or energy density) according to inputs from the user of user-selected duration or skin tone (e.g., increasing power, irradiance, time, and/or energy density for darker skin tones and/or durations lower than recommended or default for the therapy program module 522a-n). For example, the controller 124 of the therapy output device 120 is configured to operate the light sources 122 according to instructions of a therapy program module 522a-n to output the electromagnetic radiation at a first irradiance for a recommended duration (e.g., according to a first therapy program module 522), and upon receipt of a user-selected duration that is less than the recommended duration (e.g., via the user control device 140), the instructions executed by the controller 124 for the first therapy program module 522a are adjusted (e.g., by the user control device 140, such as with therapy program distribution module 542a, or by the therapy output device 120) to operate the light sources 122 to output the electromagnetic radiation at a second irradiance that is higher than the first irradiance for the user-selected duration.

The user input module 542b is configured to receive user inputs, for example, via a graphical user interface. User inputs may include, for example, selection of a therapy program, desired duration for a therapy program, biological information (e.g., skin tone), requested duration (e.g., if different than a recommended duration), and operational instructions for a therapy program (e.g., start, pause, end). The output device control module 542c is configured to send (e.g., transmit) instructions to the therapy output device 120 for operation thereof according to user inputs received by the user input module 542b, such as the selection of a therapy program, duration of the therapy program, and start, pause, or end commands for the therapy program. The usage information module 542d is configured to receive the usage information (e.g., from the therapy output device 120) and the user feedback information (e.g., from user inputs to the user control device 140), and send (e.g., transmit) the usage information and the user feedback information to the one or more servers 502. The control application 542 may also include a user-defined therapy program module 542e, which is configured to receive user inputs by which a user defines a custom therapy program module 524 according to the parameters of light sources (or wavelength), power (or irradiance), and time (or energy density). The user-defined therapy program module 542e is configured to receive inputs that correspond to the capabilities of the therapy output device 120 (e.g., wavelengths, power or irradiance at the wavelengths, and/or time or energy density) and/or other limitations (e.g., defined dosage limits). While described as separate modules, it should be understood that the various functions of the different modules described above may be combined into fewer modules and/or divided into further modules.

The therapy output device 120 is configured to receive one or more of the therapy program modules 522a-n and execute the therapy program modules 522a-n to output the electromagnetic radiation according thereto, and may be further configured to report information (e.g., usage information) back to the user control device 140 and/or the servers 502 and may be further configured to adjust output of the electromagnetic radiation according to sensed distance. For example, the therapy output device 120 includes a therapy program receiving module 526a and a program execution module 526c, and may further include a therapy reporting module 526b, and a distance adjustment module 526d. The therapy program receiving module 526a includes instructions that are executable to receive the therapy program modules 522a-n and/or the custom therapy program modules 524 from the user control device 140, which may include parameters and/or instructions according to which the controller 126 operates the light sources 122. In one example, the therapy program modules 522a-n and/or the custom therapy program module 524 include parameters (e.g., wavelength or light sources, power or irradiance of the wavelengths or light sources, and time or energy density of the wavelengths or light sources), while the therapy output device 120 includes the program execution module 526c that includes instructions by which the parameters (or proxies thereto) are received and the light sources operated according thereto. The therapy output device 120, for example, with the therapy program receiving module 526a, may be configured to store one or more of the therapy program modules 522a-n for later execution thereof (e.g., storing the most recent one or a default therapy program module that may be initiated by the therapy output device 120 itself), or may be configured to require receiving one of the therapy program modules 522a-n from the user control device 140 before each instance of starting a therapy program (e.g., when receiving an input from the user on the user control device 140).

The therapy output device 120 may further include the distance adjustment module 526d that includes instructions by which the distance between the therapy output device 120 and the user is measured with the sensor 124 and input to the program execution module 526c. The program execution module 526c is further configured to receive the distance and operate the light sources according the parameters (e.g., according to the instructions to achieve the parameters) and the distance (e.g., increasing power to achieve the irradiance parameter if the distance increases).

The therapy reporting module 526b includes instructions that are executed to send information to the user control device 140 pertaining to the therapy program modules 522a-n and the custom therapy program module 524 executed thereby, such as duration, distance, and/or completion. As a result, the user control device 140 may track use of the user control device 140 against a therapy regimen that may, for example, require a total number of treatments (e.g., number of times the therapy program module 522a-n is performed), total duration, and/or total energy density.

While described as separate modules, it should be understood that the various instructions and/or functions of the different modules described above may be provided in different manners, for example, being combined into fewer modules and/or divided into further modules. For example, as an alternative to including the parameters, the therapy program modules 522a-n and the custom therapy program module 524 may themselves include instructions that incorporate the parameters and by which the light sources are operated and/or by which the distance is measured and there light sources operated according thereto, thereby omitting the program execution module 526c and/or the distance adjustment module 526d.

As has been described previously, each of the therapy program modules 522a-n be configured to provide an intended or expected therapeutic function to the user, while the custom therapy program module 524 is defined by the user for an intended result. Each of the therapy program modules 522a-n, where n is an integer greater than one, differ from each other by one or more of the parameters (e.g., light sources and/or peak wavelength, power and/or irradiance for each light source or peak wavelength, and/or time and/or energy density of each light source or peak wavelength).

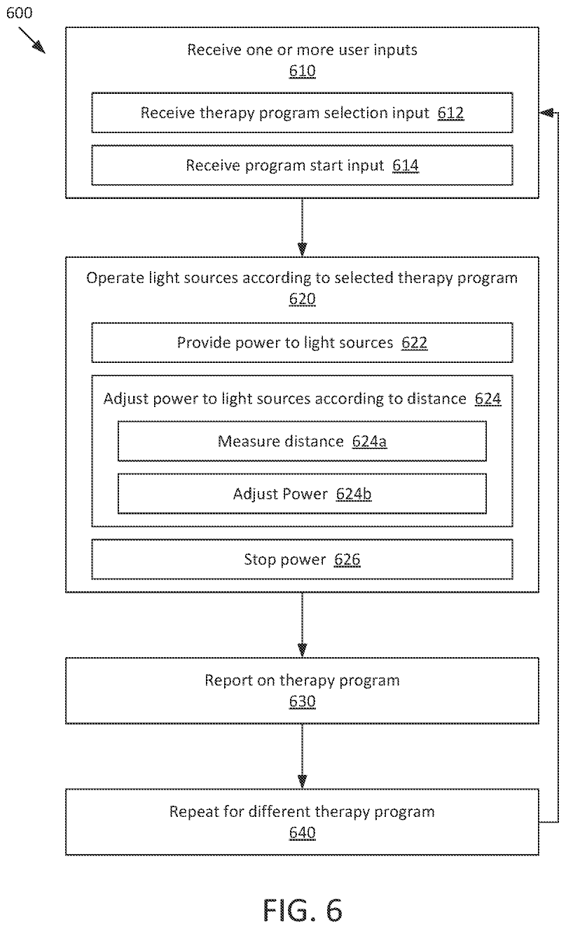

Referring to FIG. 6, a flowchart is illustrated for a method 600 of providing therapy with a therapy output device, such as providing photobiomodulation therapy with the therapy output device 120. The method 600 generally includes receiving 610 one or more user inputs of a therapy program with the output device, operating 620 light sources of the output device according to the user inputs. The method 600 may further include reporting 630 on the therapy program, and repeating 640 the receiving 610, the operating 620, and the reporting 630 (if performed) at a subsequent time for another therapy program.

The receiving 610 of the one or more user inputs with the output device may be performed, for example, with a user input of the output device (e.g., the user input 130 of the therapy output device 120) and/or a communications interface of the output device (e.g., the communications interface 226d of the controller 126 of the therapy output device 120). For example, the receiving 610 of the one or more inputs may include receiving 612 a program selection input, which is a selection by the user of a therapy program (e.g., one of the therapy program modules 522a-n or the custom therapy program modules 524). The program selection input may be received from a control device, such as a signal received from the user control device 140, that is in communication with the therapy output device 120 or may be received directly by the output device (e.g., the user input 130 of the therapy output device 120). The receiving 610 of the one or more inputs may further include receiving 614 a program start input, which is an input by the user to begin operation of the selected therapy program.

The operating 620 of the light sources of the output device according to the user inputs is controlled by a controller or other processor, such as the controller 126, according to the instructions and/or parameters of the therapy program. The operating 620 generally includes providing power 622 to one or more of the light sources, such as the light sources 122 of the therapy output device 120, to output electromagnetic radiation according to parameters of the therapy program (e.g., light sources and/or wavelengths, power and/or irradiance, and time and/or energy density). The operating 620 may further include adjusting the power 624 provided to the one or more light sources according to a distance, thus including sub steps of measuring 624a a distance between the output device and the user with a sensor, such as the sensor 124 of the therapy output device 120, and adjusting 624b the power provided to the light sources according to the distance (e.g., to achieve the parameter of the irradiance). The measuring 624a of the distance and the adjusting 624b of the power may be performed continually over the time of the therapy program. The operating 620 further includes stopping 626 providing power after the providing the power 622 and/or adjusting the power 624 after the time of the therapy program has elapsed.

The reporting 630 on the therapy program is performed by a controller or other processor, such as by the controller 126 and the communications interface 226d. The reporting 630 on the therapy program includes sending information about use and/or operation of the therapy program by the output device, for example, sending to the control device information about distance (between the output device and the user), duration (e.g., time that the therapy program was performed), and/or completion (e.g., if the therapy program was completed).

The repeating 640 of the receiving 610, the operating 620, and/or the reporting 630 is performed at a subsequent time and may be for a different therapy program. The different (e.g., another or a second) therapy program includes one or more different parameters that may include, but are not limited to, wavelength and/or light sources, power and/or irradiance, and/or time and/or energy density.

In addition to the foregoing description and the claims below, the present disclosure includes the following embodiments:

Embodiment 1. A therapy output device comprising:

a plurality of light sources that are configured to output electromagnetic radiation at different peak wavelengths; and

a controller that is configured to operate the light sources to output the electromagnetic radiation according to a first therapy program and a second program, each of the first therapy program and the second therapy program parameters according to which the controller causes the light sources to output the electromagnetic radiation to differ between the first therapy program and the second therapy program by at least one of peak wavelength, power, irradiance, time, or energy density.

Embodiment 2. A method for providing photobiomodulation therapy with a therapy output device, comprising:

receiving, with the therapy output device, a first user input selecting a first therapy program from a plurality of therapy programs, wherein the first therapy program includes parameters according to which electromagnetic radiation is output by the therapy output device with a peak wavelength, an irradiance, and an energy density, and each of the other therapy programs of the plurality have one or more different parameters according to which electromagnetic radiation is output by the therapy output device with at least one of a different wavelength, a different irradiance, or a different energy density than the first therapy program;

receiving, with the therapy output device, a second user input to start the first therapy program; and

operating the therapy output device to output electromagnetic according to the parameters.

Embodiment 3. The method according to embodiment 3, further comprising measuring, with a sensor of the therapy output device, a distance between the therapy output device and a user, and adjusting the irradiance according to the distance.

Embodiment 4. The method according to embodiment 3, wherein the receiving of the first user input includes receiving a signal from a user control device that is in wireless communication with the therapy output device.

Embodiment 5. A therapy output device comprising:

a head comprising light sources that include first light sources that output electromagnetic radiation at a first peak wavelength in a red light spectrum and second light sources that output electromagnetic radiation at a second peak wavelength in an infrared light spectrum; and

an arm having a first end coupled to the head and a second end, the arm being flexible to move the head relative to the second end and sufficiently rigid to support the head statically relative to the second end.

Embodiment 6. The therapy output device according to claim 5, wherein the arm is a gooseneck.

Embodiment 7. The therapy output device according to Embodiment 6, further comprising a base, the second end of the arm is coupled to the base, and the base is configured to rest on a flat surface and support the head and the arm thereabove.

Embodiment 8. The therapy output device according to Embodiment 5, further comprising a controller and a communications interface, wherein the communications interface is configured to receive a therapy program module from another device, and the controller is configured to operate the first light sources and the second light sources to output the electromagnetic radiation according to the therapy program module.

Embodiment 9. The therapy output device according to Embodiment 5, further comprising a grounding subsystem that, when touched by a user, conductively couples the user to ground.

Embodiment 10. The therapy output device according to claim 5, wherein the light sources include third light sources that output electromagnetic radiation at a third peak wavelength in the infrared light spectrum between 1040 and 1060 nanometers.

Embodiment 11. A therapy output device comprising:

a controller;

light sources that include one or more first light sources configured to output electromagnetic radiation at a first peak wavelength of between 650 and 670 nanometers, one or more second light sources configured to output electromagnetic radiation at second peak wavelength of between 800 and 820 nanometers, one or more third light sources configured to output electromagnetic radiation at a third peak wavelength of between 840 and 860 nanometers, and fourth light sources configured to output electromagnetic radiation at a fourth peak wavelength of between 1040 and 1060 nanometers;

wherein the controller operates the light sources according to first instructions of a first therapy program.

Embodiment 12. The therapy output device according to claim 11, wherein the controller is configured to receive second instructions of a second therapy program that differs from the first therapy program, and operates the light sources according to the second instructions.

While the disclosure has been described in connection with certain embodiments, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A therapy system comprising:
a therapy output device comprising:
a plurality of light sources that are configured to output electromagnetic radiation at different peak wavelengths; and
a controller that is configured to operate the light sources to output the electromagnetic radiation to differ between a first therapy program and a second therapy program by at least one of the peak wavelengths, irradiance of one or more of the peak wavelengths, or energy density of the one or more peak wavelengths.

2. The therapy system according to claim 1, wherein the therapy output device is configured to communicate wirelessly with a user control device, and is configured to receive instructions from the user control device for the first therapy program and the second therapy program according to which the controller operates the light sources to output the electromagnetic radiation.

3. The therapy system according to claim 2, wherein the user control device receives the instructions for the first therapy program and the second therapy program from a server.

4. The therapy system according to claim 3, wherein the therapy output device receives updated instructions for the first therapy program from one of the server or the user control device, and the controller operates the light sources according to the updated instructions to output the electromagnetic radiation to different from the first therapy program by at least one the different peak wavelengths, the irradiance of the one or more of the different peak wavelengths, or the energy density of the one or more the different peak wavelengths.

5. The therapy system according to any of claim 3, wherein the server is configured to receive usage information from at least one of the therapy output device or the user control device, wherein the usage information pertains to usage of the therapy output device by a user.

6. The therapy system according to claim 3, further comprising the server and the user control device.

7. The therapy system according to claim 1, wherein the controller is configured to operate the light sources according to the first therapy program to output the electromagnetic radiation at a first irradiance for a recommended duration, the therapy system is configured to receive a user-selected duration from a user that is less than the recommended duration, and the controller is further configured to operate the light sources according the first therapy program and the user-selected duration to output the electromagnetic radiation at a second irradiance that is higher than the first irradiance for the user-selected duration.

8. The therapy system according to claim 1, wherein the different peak wavelengths include at least three peak wavelengths selected from a group consisting of a first peak wavelength of between 650 and 670 nanometers, a second peak wavelength of between 800 and 820 nanometers, a third peak wavelength of between 840 and 860 nanometers, and, a fourth peak wavelength of between 1040 and 1060 nanometers.

9. The therapy system according to claim 8, wherein the different peak wavelengths include the first peak wavelength, the second peak wavelength, the third peak wavelength, and the fourth peak wavelength.

10. The therapy system according to any of claim 8, wherein the light sources are light-emitting diodes that each have only one of the peak wavelengths.

11. The therapy system according to claim 1, further comprising a grounding subsystem configured to conductively couple a user to ground, the grounding subsystem including a user contact configured to electrically couple to a user, a grounding contact configured to electrically couple to the ground, and a conductive path extending therebetween the user contact and the grounding contact.

12. The therapy system according to claim 11, wherein the conductive path extends through the therapy output device.

13. A therapy output device comprising:
a head comprising light sources that include first light sources that output electromagnetic radiation at a first peak wavelength in a red light spectrum and second light sources that output electromagnetic radiation at a second peak wavelength in an infrared light spectrum; and
an arm having a first end coupled to the head and a second end, the arm being flexible to move the head relative to the second end and sufficiently rigid to support the head statically relative to the second end.

14. The therapy output device according to claim 13, wherein the arm is a gooseneck.

15. The therapy output device according to claim 14, further comprising a base, the second end of the arm is coupled to the base, and the base is configured to rest on a flat surface and support the head and the arm thereabove.

16. The therapy output device according to claim 13, further comprising a controller and a communications interface, wherein the communications interface is configured to receive a therapy program module from another device, and the controller is configured to operate the first light sources and the second light sources to output the electromagnetic radiation according to the therapy program module.

17. The therapy output device according to claim 13, further comprising a grounding subsystem that, when touched by a user, conductively couples the user to ground.

18. The therapy output device according to claim 13, wherein the light sources include third light sources that output electromagnetic radiation at a third peak wavelength in the infrared light spectrum between 1040 and 1060 nanometers.

19. A therapy output device comprising:
a controller;
light sources that include one or more first light sources configured to output electromagnetic radiation at a first peak wavelength of between 650 and 670 nanometers, one or more second light sources configured to output electromagnetic radiation at second peak wavelength of between 800 and 820 nanometers, one or more third light sources configured to output electromagnetic radiation at a third peak wavelength of between 840 and 860 nanometers, and fourth light sources configured to output electromagnetic radiation at a fourth peak wavelength of between 1040 and 1060 nanometers;
wherein the controller operates the light sources according to first instructions of a first therapy program.

20. The therapy output device according to claim 19, wherein the controller is configured to receive second instructions of a second therapy program that differs from the first therapy program, and operates the light sources according to the second instructions.
